(19) United States
(12) Patent Application Publication
(10) Pub. No.: US 2012/0329125 A1
(43) Pub. Date: Dec. 27, 2012

(54) GROUP CULTURE SYSTEM AND METHOD WITH HELPER EMBRYOS

(75) Inventors: Fuliang Du, Navasota, TX (US); Giorgio Antonio Presicce, Navasota, TX (US); Juan Moreno, Navasota, TX (US); Jie Xu, Centreville, VA (US); Shinn-Chin Wu, Navasota, TX (US)

(73) Assignee: INGURAN LLC., Navasota, TX (US)

(21) Appl. No.: 13/168,707

(22) Filed: Jun. 24, 2011

(51) Int. Cl. C12N 11/10; C12N 11/04; C12N 5/00; C12M 3/00

(52) U.S. Cl. 435/178; 435/289.1; 435/174; 435/373

(57) ABSTRACT

Methods and systems for physically separating helper embryos from desired embryos in a group culturing technique in order to maintain the pedigree and genetic information of the desired embryos from different species, including cattle and human, and to provide the developmental benefits of group culturing. The separation of the groups of embryos can be the result of embedding one group of embryos in a gel or solid, or the groups can be physically separated by a membrane or other structure.

### FIELD

The present embodiments generally relate to the development of embryos and, more particularly, to the development of embryos in group cultures.

### BACKGROUND

Various artificial reproductive technologies employ a number of techniques for retrieving oocytes or eggs for fertilization, such as in vitro fertilization, and each technique possess different drawbacks. Particular problems with different collection techniques are evident in group culturing methods. In the case of bovine, large numbers of oocytes can be collected from slaughterhouse ovaries, which involves the collection and grading of cumulus-oocyte-complexes (COCs) from slaughterhouses. The anonymous collected ovaries can then be sliced and flushed for the collection of immature oocytes. However, bovine are often bred specifically for milk production or as beef cattle and reproductive cells are preferred from animals with desirable genetic pedigrees. The pedigree and genetic characteristics associated with oocytes collected from a slaughterhouse are completely unknown, presenting an unattractive option for breeding because there is no assurance an embryo will possess good genetics or desirable genetic traits.

Ovum pick up (OPU), is an ultrasound guided technique for collecting COCs from the ovaries living animals, such as bovine, and is described by Pieterse et al. in Transvaginal ultrasound guided follicular aspiration of bovine oocytes. Theriogenology 1991; 35:19-24, incorporated herein by reference. The live donors provide known genetics and pedigree, which can easily be tracked with their reproductive cells. In this way, oocytes can be procured from elite donors with superior genetics. Unfortunately, in the case of bovine, OPU sessions produce a limited number of COCs. Some bovine produce an average of eight or more (Bos taurus or Bos indicus, or derivative hybrids) oocytes per OPU session and other species and/or breeds have been shown to produce fewer COCs per session. (Merton et al. Factors affecting oocyte quality and quantity in commercial application of embryo technologies in the cattle breeding industry. Theriogenology 2009; 72:885-93, and Lopes et al., Effect of days post-partum, breed and ovum pick-up scheme on bovine oocyte recovery and embryo development. Reprod Domest Anim 2006; 41:196-203, each incorporated herein by reference). These low numbers of oocytes can be too few for certain reproductive techniques, particularly group culturing of embryos. (Keefer at al. In vitro culture of bovine IVM-IVF embryos. Cooperative interaction among embryos and the role of growth factors. Theriogenology 1994; 41:1323-31 and O'Doherty et al. Effects of culturing bovine oocytes either singly or in groups on development to blastocysts. Theriogenology 1997; 48:161-69, each incorporated herein by reference.)

Group culturing provides mutually beneficial effects on developing embryos in mice (Canseco et al., Embryo density and medium volume effects on early murine embryo development. J Assis Reprod Genet 1992; 9:454-57), sheep (Gardner et al. Enhanced rates of cleavage and development for sheep zygotes cultured to the blastocyst stage in the absence of serum and somatic cells: amino acids, vitamins, and culturing embryos in groups stimulate development. Biol Reprod 1994; 50:390-400), and cattle (Khurana et al. Effects of oocyte quality, oxygen tension, embryo density, cumulus cells and energy substrates on cleavage and morula/blastocyst formation of bovine embryos. Theriogenology 2000; 15:741-56, each incorporated herein by reference). This could be due to the embryotrophic factors produced when a number of embryos are in close proximity (Gopichandran et al. The effect of paracrine/autocrine interactions on the in vitro culture of bovine preimplantation embryos. Reproduction 2006; 131:269-77, incorporated herein by reference). COCs collected from slaughterhouse ovaries can be undesirable for this technique because the donors are anonymous. In order to preserve the pedigree and genetic information provided by COCs collected by OPU, the oocytes and developing embryos of each donor must be cultured separately and sessions of OPU can fail to produce sufficient numbers of COCs for group culturing. Therefore, a need exists for an improved method of group culturing embryos operating within the limitations of current oocyte collection techniques. A need further exists for an improved method of group culturing embryos which maintains the genetic and pedigree information of embryos being cultured.

Still a further need exists for an improved method of group culturing for a limited number of embryos produced from sex selected sperm.

### SUMMARY OF THE INVENTION

Certain embodiments described herein meet the needs set forth above and relate to a method and system of group culturing desired embryos in the proximity of helper embryos in order to improve the development of the desired embryos.

Certain embodiments relate to methods and systems for physically separating helper embryos from desired embryos in order to maintain the pedigree information of the desired embryos and to provide the developmental benefits of group culturing for the desired embryos. The method can include the steps of obtaining at least one embryo; obtaining at least one helper embryo; and culturing the at least one embryo with the at least one helper embryo. Separation can be maintained between the at least one helper embryo and the at least one embryo during the step of culturing by embedding the at least one helper embryo in a gel or solid suspension. The suspension can then be cultured in close proximity to the at least one embryo. The suspension can comprise an agarose chip permeable to supporting and promoting chemical factors. The helper embryos can be embedded in the agarose chip by: providing a solution containing agarose; melting the solution; adding at least one helper embryo to the melted solution; and aspirating the at least one helper embryo and melted agarose solution to form a chip.

Another embodiment relates to a method of culturing embryos by collecting a first group of oocytes; fertilizing the first group of oocytes to form a group of helper embryos; collecting a second group of oocytes; fertilizing the second group of oocytes with sex sorted sperm to form a group of sorted embryos; and culturing the helper embryos with the sorted embryos wherein the groups of embryos are physically separated.

Some embodiments described herein relate to a method of embedding helper embryos in an agarose chip so the helper embryos can be cultured with desired embryos promoting the development of the desired embryos while remaining physically separate from the desired embryos. A method for embedding embryos in an agarose chip can include the steps of producing a solution with agarose and NaCl; autoclaving the solution; heating the solution to melt the agarose; cooling the solution to about a normal temperature for embryos; adding embryos into the solution; aspirating the embryos and agarose solution into an instrument with a channel; and releasing the embryos and agarose solution on a cooler surface for solidifying the agarose.

Additional embodiments relate to containers or systems such as Petri dishes with wells or other incubation spaces for maintained embryos in close proximity to helper embryos. The wells or other incubation spaces can contain helper embryos, which can be separated from additional embryos by membranes, or other porous materials such as mesh. The system can include at least one well and a plurality of helper embryos within the well or wells. The helper embryos can be embedded within each respective well or physically separated in a portion of the well by a membrane, a mesh or another permeable divider.

### BRIEF DESCRIPTION OF THE DRAWINGS

FIG. 1A-1B illustrates a container in accordance with certain embodiments of the present invention.

FIG. 2A-2F illustrate various embodiments of wells in accordance with certain embodiments of the present invention.

FIG. 3 illustrates a flow diagram of a method in accordance with certain embodiments of the present invention.

FIG. 4A-4D illustrates group culturing embryos in accordance with certain embodiments of the present invention.

### DETAILED DESCRIPTION THE PREFERRED EMBODIMENTS

Certain embodiments relate to a method of developing embryos in group cultures. The embryos can be from any mammalian species, including, but not limited to, bovine, equine, porcine, cervine, human, sheep, goat and other species. The method can include obtaining at least one embryo. The at least one embryo can interchangeably be referred to as a desired embryo, a free floating embryo, or a targeted embryo in various embodiments and should be understood to be the target embryo or group of target embryos for development. The at least one embryo can be produced from oocytes fertilized in vitro, by an ICSI (intracytoplasmic sperm injection) technique, or by other know fertilization methods. The at least one embryo can also be obtained thought nuclear transfer procedure, such as a cloned embryo. The embryo can also be obtained thought chemical activation, physical activation or a combination of both of an oocyte, resulting in a parthenogenetic embryo. In one embodiment, the embryo can be produced by fertilizing an oocyte with sex sorted sperm. Specific examples are given regarding embryos, but zygotes or fertilized eggs may be used prior to cleavage in some embodiments.

The oocyte or oocytes used to produce the desired or targeted embryo can originate from a pedigreed mammal with desirable genetic characteristics or from an anonymous donor. The oocytes can be obtained through an OPU procedure, through flushing, through collection from slaughterhouse ovaries, or by other means known in the art for collecting oocytes, or COCs, or derivative of stem cells, or derivative of induced pluripotent stem cells (iPS). OPU can be performed as described by Pieterse et al. in Transvaginal ultrasound guided follicular aspiration of bovine oocytes. (Theriogenology 1991; 35:19-24), the entire text of which is incorporated herein by reference. Additional helper embryos can be produced from oocytes acquired in any of the same ways described above.

Helper embryos can be embryos from the same species or from different species compared to the at least one embryo, or the targeted embryo. For example, rabbit helper embryos can be used to promote the development of targeted bovine embryos.

The helper embryos can be obtained by fertilizing oocytes from anonymous donors or from a pedigreed animal such as a bovine with desirable genetic characteristics. The helper embryos can be obtained by fertilizing oocytes with sorted sperm, such as sex sorted sperm, or with conventional un-sorted sperm. The helper embryo or embryos can also be obtained through nuclear transfer procedure. The helper embryos can also be obtained thought chemical activation of an oocyte, physical activation or an oocyte or a combination of both resulting in parthenogenetic embryos.

The oocytes used to produce helper embryos can be obtained through an OPU procedure, through flushing, through collecting from slaughterhouse ovaries, or by other means known in the art for collecting oocytes or COC, or derivative of stem cells, or derivative of induced pluripotent stem cells (iPS). The helper embryos can be lacking in pedigree or particular genetic characteristics of interest and can be obtained from gametes of anonymous donors. However, helper embryos are not limited to embryos produced from one or more anonymous donors.

However each group of embryos is obtained, the helper embryos can then be cultured with the at least one embryo, or the targeted embryos. All the embryos can be cultured in a space, such as a well, and can be collected in a fluid volume of between about 100 μL and 1 μL. The helper embryos can physically be separated or segregated from the at least one embryo by a membrane, other porous structure, or can be embedded in a permeable suspension. Other methods of separating the targeted or desired embryos from the helper embryos are contemplated beyond these specific examples. Such a separation only needs to be a physical separation so the desired embryos can be identified and collected to maintain the integrity of their pedigree and genetic characteristics. The separation can allow biochemical and physiological exchanges to and from the helper embryos so that promoting factors, embryotrophic factors, and other chemicals benefiting embryo development are exchanged between the two groups. For example, the helper embryos can be embedded in a gel or solid suspension wherein the suspension is cultured with the at least one embryo. The gel or solid would generally have permeability to embryotrophic factors produced by the both groups of embryos.

In one embodiment, the suspension can comprise an agarose chip. The agarose chip can be formed by melting a solution containing agarose, adding helper embryos to the melted solution; and aspirating the helper embryos and melted agarose solution to form a chip. In another embodiment, the helper embryos can be separated from the remaining embryos by a membrane, a mesh material or a porous layer. The present invention contemplates any suitable material can be used which sufficiently physically separates the helper cells, while remaining permeable for the various promoting factors and embryotrophic factors to provide group culturing benefits.

In yet another embodiment, the combined total embryos from the helper embryos and the at least one embryo in a group culture can be at least, but not limited to, ten embryos. In yet another embodiment, the total number of embryos in a group culture can be about 20 to about 40 embryos. In an alternative embodiment, where the group culture occurs in a smaller volume of medium, 1-10 μL for example, as few as a single embryo could be cultured with a single helper embryo. The referenced embryo totals can be formed with any combination of helper embryos. By way of a non-limiting example, one to nine or more helper embryos can be used for a group culture of ten total embryos. Similarly, one to nine or more of the desired embryos can be used for a group culture of ten total embryos.

In one embodiment, the helper embryos are kept between about 2° Celsius and about 45° Celsius after being embedded in the solid or gel suspension.

One embodiment relates to a culturing container with incubation spaces, such as wells, for culturing groups of embryos. The culturing container can be a Petri dish or another container with a generally flat bottom surface. The culturing container can include at least one well on the bottom surface. Helper embryos can be placed in the well and either embedded in an agarose chip, or contained within the well by a membrane. In one embodiment, the well is divided with a divider for separating helper embryos. In another embodiment, the well can be divided into two concentric areas. The helper cells can be placed in either of the inner or outer area, and the remaining embryos, the embryos targeted for development, can be placed in the remaining area.

In one embodiment, each well can further comprise a raised portion. The raised portions can further comprise inner wells. The helper embryos can then be stored in the inner wells and embedded in a material, or separated with a membrane. An advantage in embedding the helper embryos in contrast to the remaining targeted embryos resides in the extra stresses and process steps, including temperature changes, embedded embryos endure. In embodiments including membranes, meshes, or porous barriers, the helper embryos and the remaining targeted embryos can interchangeably be placed on either side as long as each group is physically separated.

Other methods of physically separating the desired embryos from the helper embryos are contemplated beyond these specific examples. Such a separation only needs to be a physical separation such that desired embryos can be identified and collected in order to maintain the integrity of their pedigree and genetic characteristics. The separation must further allow biochemical and physiological exchanges to and from the helper embryos so promoting factors, embryotrophic factors, and other chemicals benefiting embryo development are exchanged between the two groups. For example, the helper embryos can be embedded in a gel or solid suspension and wherein the suspension is cultured with the at least one embryo. A low melting point agarose can be used to create an agarose chip in which helper embryos can be embedded.

In one embodiment, an agarose chip can be formed by producing a solution with agarose and NaCl. The agarose can have a gelling point and a low melting point and make up roughly 1-20% of the solution. The solution can be sterilized by autoclaving and then heated to 65° Celsius or higher in order to melt the agarose. The melted agarose can then be cooled to a suitable temperature for embryos. In the case of bovine, the solution can be cooled to between about 35° Celsius and 45° Celsius, and more particularly can be cooled to 39° Celsius. However, this method is contemplated for use with bovine, equine, porcine, cervine, human, sheep, goat and other species. The solution would be cooled to the appropriate temperatures for any given species.

Once at the appropriate temperature, embryos can be added to the solution. In one embodiment, the embryos can be in the two to eight cell stage of development. In another embodiment, embryos can be used with more than eight cells. And in yet another embodiment, oocytes, zygotes, or fertilized eggs, can be used before cleavage. The solution, including the embryos, can be aspirated into an instrument with a channel, such as a pipette. The desired number of embryos can be aspirated with the surrounding agarose solution, and then released into a cooler medium between about 2° Celsius and 35° Celsius. This cooler temperature will solidify the agarose solution around the embryos embedding the embryos in a sausage shaped agarose chip.

This agarose chip provides a sufficient means to separate the embedded helper embryos, with any embryos outside the chip in order to prevent mixing. This chip also allows chemicals to exchange between the separated groups of embryos, including embryotrophic factors.

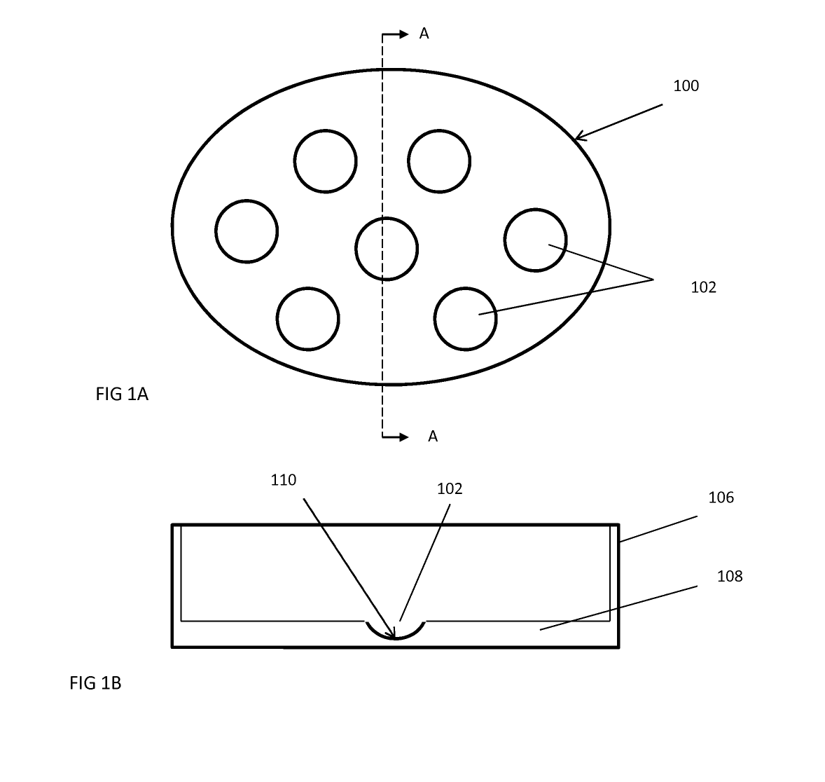

Turning now to the figures, and specifically to FIGS. 1A and 1B, a container 100 is illustrated with a plurality of wells 102. The container 100 can serve as a culturing system for culturing helper embryos alongside targeted embryos. Cross section AA is taken through the center of the container 100 and provides a view of the sidewall 106 and the bottom 108. Additionally, a bottom surface 110 can be seen in the well 102. Such a container 100 can be a Petri dish or another container with a sufficient bottom for the inclusion of wells 102. The plurality of wells 102 can each accommodate helper embryos, which can be embedded within the wells 102, or can be embedded in an agarose chip or other gel, which is placed into the wells 102.

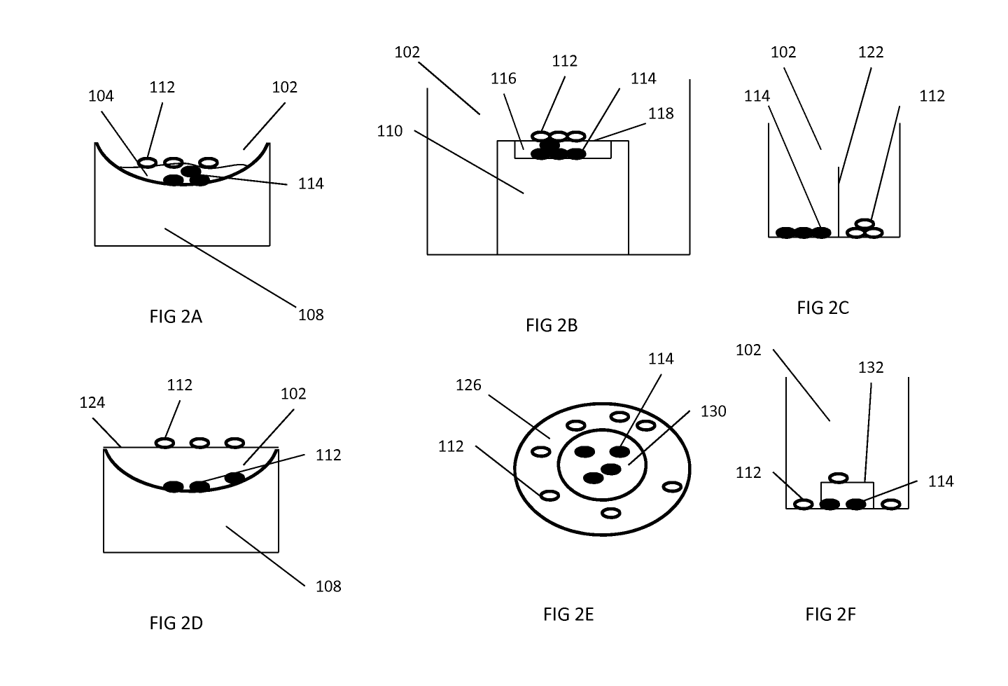

FIG. 2A-2F illustrate several configurations within the well 102 for placing helper embryos 114 in the vicinity of at least one targeted embryo 112, while maintaining a physical separation between the two groups of embryos. FIG. 2A illustrates a cross section of a well 102 with helper embryos 114 embedded in a gel or solid 104 on the bottom surface of the well 102 formed in the bottom 108 of a container. The targeted embryos 112 are placed above the solid or gel 104 and can be in a free floating relationship. The targeted embryos 112 can include at least one embryo having desirable genetic characteristics and/or a know pedigree, and may interchangeable be referred to as the desirable embryos. The solid or gel 104 can be an agarose material within which the helper embryos 114 are embedded.

FIG. 2B illustrates another embodiment of a well 102 having a raised portion 110. The raised portion 116 can have an inner well 116 capable of holding cells, such as helper embryos 114. The helper embryos 114 can be physically separated from the targeted embryos 112 with a membrane 118 covering the inner well 116. The membrane 118 should maintain separation between the two groups of embryos while permitting promoting factors to exchange between the groups.

FIG. 2C illustrates another embodiment of the well 102, where a membrane or porous barrier 122 divides the well 102 horizontally. The membrane 122 is illustrated roughly in the center of the well 102, but the well 102 can be divided asymmetrically for the helper embryos 114 and the targeted embryos 112. The membrane or porous barrier 122 functions to separate the groups of cells while chemicals, including promoting factors, are still exchanged between the groups of embryos.

FIG. 2D illustrates a close up view of a well 102 having a membrane 124 across the surface of the well 102. Helper embryos 114 can be placed in the well 102, and then the well can be isolated with a membrane 124. Subsequently, at least one targeted embryo 112 can be place on the membrane in the proximity of the helper embryos 114. The at least one targeted embryo 112, or desirable embryo, can be placed into the well 102 and sealed with a membrane 124 with helper embryos 114 subsequently placed over the membrane 124, as long as each group of cells in maintained separated, but in close physical proximity.

FIGS. 2E and 2F illustrate an embodiment where each of the helper embryos 114 and the targeted embryos 112 are placed in the bottom of a well 102. The helper embryos 114 can be confined in a first region 130 adjacent to the targeted embryos 112 confined in the second region 126. The first region 130 can be defined by a barrier 132. The barrier 132 can be walls covered by a membrane, a porous structure, or even a solid or a gel. By way of an example, the barrier can be an agarose chip within which the helper embryos 114 are embedded. A variety of alternatives are envisioned with these configurations. These figures represent illustrative examples, the features of which can be combined within the scope of the present invention.

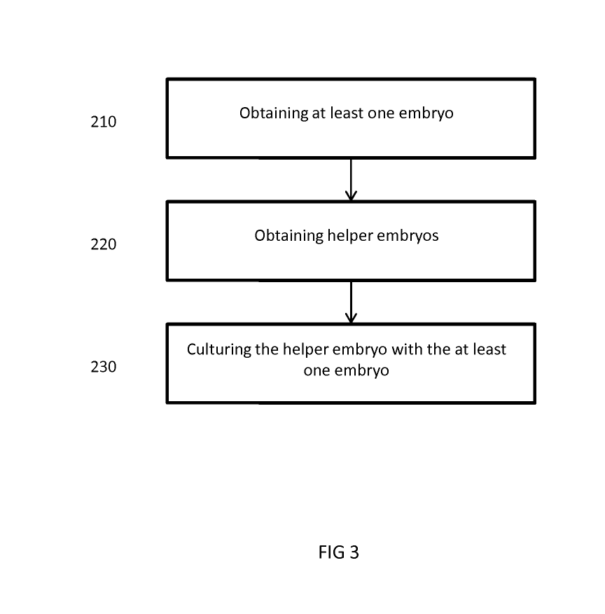

FIG. 3 illustrates a block diagram in accordance with an embodiment of one method in accordance with the present invention. At step 210, at least one embryo is obtained, such as a targeted embryo or group of targeted embryos. As previously discussed, this embryo, or group of embryos can be selected for the desirable genetic traits of the donor. In one embodiment, an oocyte for producing the embryo can be obtained through a session of OPU on a donor. Collected oocytes can then be fertilized in vitro and monitored for cleavage. However, other methods of obtaining oocytes are envisioned within the scope of the present invention, and other techniques, such as flushing or collection from slaughter hour ovaries, can be used.

At step 220 helper embryos are obtained. Helper embryos can be obtained in any of the same ways as the embryos from step 210. Additionally, there is no particular need to maintain the identity of the donors for the oocytes used to produce helper embryos thereby allowing more cost efficient methods of collecting oocytes and producing helper embryos. For example, oocytes can be obtained from slaughterhouse ovaries, in the case of bovine. The oocytes can then be fertilized in vitro, or artificially activated, or nuclear transferred, and monitored for cleavage. Helper embryos can be the same species or different species as compared to the at least one embryo. Steps 210 and 220 can be performed in any order, or even at the same time.

At step 230, the helper embryos can then be cultured with the at least one embryo, or with the targeted embryos. In order to promote embryotrophic factors and factors which promote embryo growth the groups of embryos can be cultured in a close proximity. By way of a non-limiting example, culturing can occur in a droplet with a volume of medium of about 1-50 μL. In accordance with one embodiment of the present invention, the step of culturing the groups of embryos together can further include the step of separating the groups of embryos, and maintaining a physical separation throughout the culturing process. The physical separation of groups helps ensure the integrity of embryos created from oocytes with known donors.

Physical separation can be maintained by embedding the helper embryos in a gel or solid suspension which is permeable to various embryotrophic factors. As one example the helper embryos can be embedded in an agarose chip. Physical separation can also be maintained by a membrane, a mesh, a porous structure or another permeable barrier.

Various numbers of embryos and helper embryos can be used in the step of culturing. For example, a total of 10 or 20 embryos can be used in a 50 μL droplet. However, in small droplets, embodiments of the current invention contemplate using fewer total embryos. For example, as few as a single helper embryo might be cultured with a single embryo in a medium droplet of about 10 μL to 1 μL.

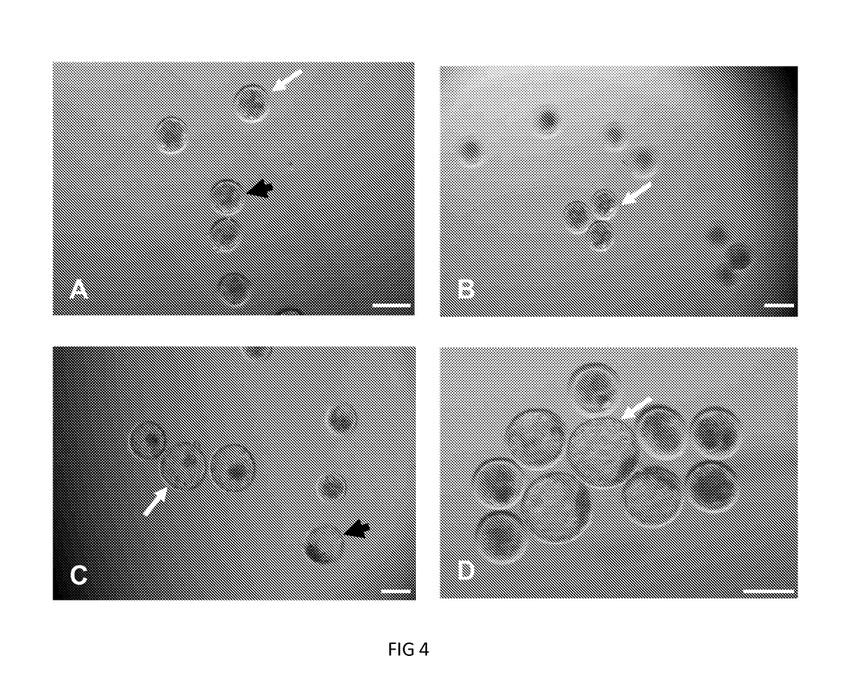

Referring now to FIG. 4A-4D, in vitro development of fertilized bovine embryos cultured in groups with agarose embedded helper embryos can be seen. FIG. 4A illustrates embryos (2-8 celled stage) embedded in 1% agarose chips after IVF, indicated by black arrow. FIG. 4A illustrates a single 1 (indicated by white arrow) embryo in the vicinity of the agarose chip, while FIG. 4B illustrates three (indicated by white arrow). Each of these embryos can be cultured, freely and separately, in a 50 μl culture droplet together with either 9 or 7 embryos embedded in an agarose chip. The total number of embryos per droplet for the embodiment illustrated is 9 and 7 respectively. After additional 5 days culture at 39° Celsius in 5% CO2, 5% 02 and 90% N2 humidified air, the embryos (OPU derived) developed into expanded blastocysts (FIG. 4C, white arrow), at a rate similar to that of the controls (FIG. 4D), in which a group of ten embryos was freely cultured in the same size droplet (50 μl). Bar=140 μm.

### EXAMPLES

### Materials and Methods

All chemicals originated from Sigma Aldrich (St. Louis, Mo.) unless otherwise noted. The COCs were matured at 39° Celsius in 5% CO2 and humidified air, and fertilized oocytes were cultured in vitro at 39° Celsius in 5% CO2, 5% O2 and 90% N2 in humidified air.

Oocytes Collected from Slaughterhouse Ovaries

Bovine COCs for in vitro embryo production were recovered from slaughterhouse Holstein ovaries and processed. Cattle ovaries were collected at a local slaughterhouse and brought to the lab within 2-3 h. No test was conducted to verify the possibility of infectious agents present in slaughterhouse material. Recovery of COCs from small to medium size ovarian antral follicles was accomplished by vacuum pump aspiration at a flow rate of 15 to 20 mL per min. The collected oocytes were graded morphologically based on the cumulus investment as follows: Grade A, >4 layers of cumulus cells; Grade B, 3 or 4 layers of cumulus cells; Grade C, 1 or 2 layers of cumulus cells; Grade D, denuded oocytes; Grade E, oocytes with expanded cumulus. To be consistent with usable COCs in Experiment 2 from OPU sessions, only COCs at grade A to C were selected for further processing. Selected oocytes were used in IVM, IVF and group culture for Experiments 1 and 2 as described below.

Animals and Oocytes Collected by OPU

Animals ranging from heifers to 6-8 yr old pluriparous Holstein cows were used for this study. They were stall fed and kept in the barn under controlled conditions. Twenty animals were used for oocyte retrieval during four replicates.

A portable Aloka 500 ultrasound unit equipped with a 5-MHz sector scanner vaginal probe (Aloka Co. Ltd, Tokyo), together with a 17-ga, 60-cm single lumen needle fitting a metallic needle guide were used for transrectal oocyte retrieval. Animals were restrained in a squeeze chute and prepared for follicular aspiration as described by Pieterse et al., Theriogenology 1991; 35:19-24, incorporated herein by reference. Aspiration medium consisted of phosphate-buffered saline (PBS) with the addition of 10 IU/mL heparin and 0.1% polyvinyl alcohol. OPU was scheduled twice weekly, a total of 4 replicates were performed. COCs of Grade A to C were selected for subsequent IVM and IVF.

Maturation, Fertilization and Culture In Vitro

Embryos were produced as described by Xu et al. Developmental potential of vitrified Holstein cattle embryos fertilized in vitro with sex-sorted sperm. Journal of Dairy Science 89:2510-18 (2006), the text of which is incorporated herein by reference. Briefly, selected COCs were matured for 22 h in 75 μL droplets of Medium 199 (Invitrogen) containing Earle's salts, L-glutamine, 2.2 g/L sodium bicarbonate and 25 mM Hepes, supplemented with 10% (vol/vol) fetal bovine serum (FBS; Hyclone, Logan, Utah), 0.5 μg/mL ovine FSH (National Institute of Diabetes and Digestive and Kidney Disease, NIDDK, Los Angeles), 5.0 ng/mL ovine LH (NIDDK), and 1.0 ng/mL estradiol 17-β. Droplets were covered with mineral oil and contained 15 to 20 oocytes.

Fertilization was accomplished by use of frozen/thawed semen from bulls of known fertility and previously tested for IVF efficiency in our laboratory. In Experiment 1 and 2, sperm was subjected to a swim-up procedure for 1 h. Following centrifugation, the sperm pellet was re-suspended to achieve a concentration of 2×106/mL. The final concentration of sperm was 1×106/mL in 50 μL droplets of TALP fertilization medium supplemented with 10 ng/mL heparin after adding both sperm and COCs. For culture, IVF droplets were covered with mineral oil, and sperm/COCs co-incubation was allowed for 20 to 22 h. Presumptive zygotes were stripped of enclosing cumulus cells by vortexing in a 0.1% hyaluronidase solution, and then moved into 50 μL droplets of culture medium consisting of synthetic oviduct fluid (SOF) medium with the addition of 6 mg/mL BSA, essential and non essential amino acids, but no serum, under mineral oil (serum free culture). Cultures were placed in a modulation chamber (Form a Scientific, USA), under a mixed gas atmosphere of CO2 (5%), O2 (5%) and balanced with N2 (90%) for an additional 20 to 24 h, for a total of 40-46 h post IVF. In Experiment 3, for IVF of OPU oocytes with X-sorted sperm, Brackett and Oliphant (BO) medium, described in Biol Reprod 1975; 2:260-74, herein incorporated by reference, was used. Briefly, straws containing sexed semen at the concentration of 8×106/mL (2×106/mL per 0.25 mL straw) were thawed for 10 s in a 37° Celsius water bath after 10 seconds of gentle shaking in air at room temperature. Sperm were washed in 8 mL of BO medium supplemented with 3 mg/mL of BSA and 10 mM caffeine and centrifuged at 1,500 g for 8 min. Sperm pellet was re-suspended and centrifuged once again, and re-suspended in BO washing medium at a concentration of 0.6×106/mL. Matured COCs were rinsed in BO medium containing 6 mg/mL BSA and 10 ng/mL heparin. Fertilization droplets (50 μL) containing matured COCs were prepared in small Petri dishes. Processed semen was added (50 μL) for a final droplet volume of 100 μL under medical oil for a final sperm concentration of 0.3×106/mL as described by Xu et al. Following 6 hours of sperm/COCs co-incubation, presumptive zygotes were moved into 50 μl culture droplets in serum free medium described above, and cultured for 40 hours (total 46 h post IVF) prior to adding helper embryos embedded in agarose chips to the culture.

Culture of Cleaved Embryos with Agarose Embedded Helper Embryos

A 1% solution of agarose, with low gelling and melting points (A-9414) was prepared in saline and then sterilized by autoclaving. Solidified agarose was stored at 2-8° Celsius prior to use. For embedding of helper embryos in agarose chips, the sterile agarose was melted by warming it in a 65° Celsius water bath, and maintaining it on a 39° Celsius warming plate until inserting helper embryos. Cleaved helper embryos at 2-8 celled stage were selected and transferred into a Petri dish containing the melted agarose at a temperature between 35 and 39° Celsius. Five, seven or nine cleaved helper embryos were aspirated into a hand-made capillary along with agarose to form a sausage-like gel (chip) (FIG. 4), and then released into culture medium at 25-30° Celsius. When solidified, the agarose/embryo chips were transferred into 50 μl droplets of SOF medium containing the free embryos or targets embryos, and culture continued for an additional 5 days. The embedded helper embryos could easily be observed within the agarose chip, and although diffusion could take place between them and the shared medium, they remained physically separated from the free or targeted embryos (FIG. 4).

### Example 1

### Determining the Minimum Number of Free Embryos to Culture with Agarose Embedded Helpers for Effective In Vitro Development: IVF with Conventional Semen

This experiment was designed to establish the minimum number of cleaved embryos that could be cultured in a single droplet without compromising their development to blastocysts. IVF was preformed with oocytes from slaughterhouse ovaries, and conventional semen, then after 40-46 hours cleaved embryos at either 1, 3, 5, 10 or 20 per group were cultured in a 50 μL medium droplet for an additional 5 days under the environmental conditions described above. The optimal number to maximize blastocyst yield was determined to be ≧10. Therefore, cleaved embryos (2-8 celled) in groups of either 1, 3 or 5, to approximate the numbers likely available from OPU/IVF, were cultured together with either 9, 7 or 5 helpers embedded in agarose chips (FIG. 1), thus, bringing the total number cultured in each droplet to 10. The results can be seen in Tables 1 and 2.

TABLE 1
In vitro development of small number of IVF embryos cultured in groups

| Treatment | Control | | | | |
|---|---|---|---|---|---|
| No. of CE per | 1 | 3 | 5 | 10 | 20 |
| droplet | | | | | |
| No. of | 62 | 27 | 27 | 39 | 24 |
| replicates | | | | | |
| Total No. of CE | 62 | 81 | 135 | 390 | 480 |
| % of blastocyst | 6.6 ± 0.4a | 11.1 ± 0.6a | 24.4 ± 1.1a | 39.2 ± 5.1b | 43.3 ± 3.4b |
| (Mean ± | | | | | |
| SEM)ψ | | | | | |

CE, cleaved embryos (2-8 celled).

TABLE 2
In vitro development of small number of IVF embryos
cultured in groups with agarose embedded helpers

| | |
|---|---|
| Treatment | Agarose embedded culture* |

| | | | |
|---|---|---|---|
| No. of CE per | 1 + 9 | 3 + 7 | 5 + 5 |
| droplet | | | |
| No. of replicates | 68 | 29 | 27 |
| Total No. of CE | 68 | 87 | 135 |
| % of blastocyst | 49.5 ± 6.0b | 41.2 ± 14b | 39.3 ± 9b |
| (Mean ± | | | |
| SEM)ψ | | | |

CE, cleaved embryos (2-8 celled).
*Cleaved targeted embryos in groups of either 1, 3, or 5 were cultured with either 9, 7, or 5 helper embryos embedded in agarose chips, respectively, in droplets containing 50 μL culture medium. The total number of embryos was 10 per droplet.
ψThe p values within the same row, and with different letters (a, b) differ (p < 0.05).

The total number of oocytes used for IVM and IVF in Experiment 1 was 3460, and 40-46 hours post IVF, 72.1% oocytes cleaved to 2-8 celled stage. Cleaved embryos (n=2388) were randomly allocated into a control or a treatment group. Controls consisted of groups of 1(n=62), 3 (n=81), 5 (n=135), 10 (n=390) or 20 (n=480) freely floating cleaved embryos, or targeted embryos, in 50 μL droplets of culture medium (Table 1). In this experiment, morula development was not examined due to the large scale of the experiment. Blastocyst development rate increased from 6.6%, to 11.1%, to 24.4% in the groups of 1, 3, or 5 embryos per droplet, respectively. However, even the best development achieved was still significantly lower than that of the group with 10 embryos per droplet (24.4% vs. 39.2%). When the number of embryos was increased to 20 per droplet, the blastocyst rate reached a plateau (43.3%) not significantly greater than achieved with 10 embryos per droplet.

Treated groups consisted of 1 (n=68), 3 (n=87), or 5 (n=135) (FIG. 1) freely floating cleaved embryos, or targeted embryos, in 50 μL droplets of culture medium with the addition of helpers of 9 (n=612), 7 (n=203), or 5 (n=135) embedded in agarose (Table 2). In these groups of 1+9, 3+7 and 5+5, the blastocyst development of the free floating embryos was 49.5, 41.2 and 39.2%, respectively, which was not significantly different from that of the controls when 10 (FIG. 4D) or 20 embryos were cultured per droplet (Table 1). In addition, the agarose embedded embryos, in these groups of 10, developed to blastocysts (FIG. 4C) at a similar rate of 38.5, 42.3 and 43.5%, respectively. There was no difference in the development between the free-floating and embedded groups or the targeted group and the helper group.

### Example 2

### Determining the Effect of Agarose on the Development of Embryos In Vitro

This experiment was performed to determine whether the addition of agarose has any effect on blastocyst development. The 2×2 experimental design was arranged to test both the number of embryos per group (3 vs. 10), and effect of agarose (with vs. without). Cleaved embryos were derived from IVF with conventional semen and matured oocytes collected from slaughterhouse ovaries. The culture was same as in Experiment 1. The results are shown in Table 3.

TABLE 3
In vitro development of small number of IVF embryos
cultured in groups with the addition of agarose chips

| Treatment* | | | | % developed to | |
|---|---|---|---|---|---|
| | No. CE/ | | No. | (Mean ± SEM) | |
| Agarose | Droplet | No. CE | Replicates | Morulae | BLs |
| With | 3 | 66 | 4 | 30.0 ± 2.51a | 27.8 ± 1.96a |
| Without | 3 | 63 | 4 | 37.8 ± 2.32a | 23.4 ± 3.64a |
| With | 10 | 100 | 4 | 46.0 ± 3.31b | 41.5 ± 5.89b |
| Without | 10 | 90 | 4 | 48.9 ± 132b | 46.7 ± 4.71b |

CE, cleaved embryos (2-8 celled).
*Cleaved embryos in groups of either 3 or 10 were cultured with or without addition of agarose chips in droplets containing 50 μL culture medium. The p values within the same column, and with different letters (a, b) differ (p < 0.05).

A total of 711 oocytes were fertilized with conventional semen, and the cleavage rate at 40-46 h after IVF reached 68.5±5.3%. The cleaved embryos were then utilized to test the effect of agarose. As shown in Table 3, the development to morula and blastocyst stage in the group of 3 per droplet was 30.0-37.8% and 23.4-27.8%, respectively, which was significantly lower than their counterparts with 10 per droplet (morula 46.0-48.9%, blastocyst 41.5-46.7%). The addition of an agarose chip did not promote embryo development to the blastocyst stage, nor did it have any detrimental effect on development.

### Example 3

### In Vitro Developmental Potential of OPU/IVF Derived Embryos when Group Cultured with Agarose Embedded Helper Embryos: IVF with X-Sorted Sperm

Oocytes were collected by OPU, and fertilized in vitro, by standard procedures described above, using X-sorted sperm. Oocytes collected from slaughterhouse ovaries, in preparation to be helper embryos, underwent the same IVM/IVF on the same time schedule as that used for OPU/sexed-IVF embryos. Seven cleaved embryos at 2-8 celled stage were embedded in each agarose chip. To test whether group culture could promote blastocyst development of OPU/sexed-IVF embryos, 46 h post IVF, the groups of 3 cleaved embryos were cultured either with or without the agarose chip containing 7 helpers (3+7 vs. 3+0) in 50 μL culture medium droplets for an additional 5 days. Therefore, the total number of embryos per culture droplet was either 10 (OPU: 3+helpers: 7) or 3 (OPU: 3+helpers: 0). The results can be seen in Table 4.

TABLE 4
In vitro development of OPU/sexed IVF embryos group-cultured with agarose embedded helper embryos

| | | | | | % developed to (Mean ± SEM)* | | | |
|---|---|---|---|---|---|---|---|---|
| | No. | No. | Agarose | No. total | 2-8 | | | |
| Group | Oocytes | Replicates | Treatment | CE/Droplet | celled | Morulae | BLs | BLs/CEs |
| OPU | 162 | 4 | 3 + 0 | 3 | 69.2 ± 3.4a | 16.9 ± 1.6a | 11.8 ± 0.9a | 17.1 ± 2.2a |
| OPU + helper | 150 | 4 | 3 + 7 | 10 | 72.3 ± 2.2a | 45.9 ± 5.4b | 37.1 ± 3.7b | 51.3 ± 3.1b |

Helper, agarose embedded embryos; BLs, blastocysts; CE, cleaved embryos (2-8 celled); OPU, Ovum pickup.
*After IVF and culture in vitro for 40 h, cleaved embryos derived from OPU/IVF in groups of 3 were cultured with either 0 or 7 helper embryos embedded in agarose chips, in droplets containing 50 μL culture medium. The number of cultured embryos per droplet was either 3 (OPU group) or 10 (OPU + helper). The cleavage to 2-8 celled stage, and development to morulae and blastocysts, except for the values of BLs/2-8 celled, were calculated based on the total number oocytes collected by OPU and used for IVF. The p values with different letters (a, b) within the same column differ (p < 0.05).

A total of 561 Grade A to C COCs were retrieved by OPU in four replicates on twenty animals. The mean number of collected oocytes was 7.1±0.45, and ranged from 3 to 20 oocytes per donor. After IVF and culture for 40 h, and prior to group culture, the cleavage rates of OPU (69.2%) and OPU-helpers (72.3%) were similar (Table 4). Cleaved embryos were randomly assigned to control (OPU: 3+helpers: 0) and treatment (OPU: 3+helpers: 7) groups. Following culture with agarose embedded helpers, the overall development rate to blastocyst was significantly higher in the OPU-helper group (3+7, 37.1%) (FIG. 1 B, C) compared with OPU group (3+0, 11.8%) (Table 4). When the blastocyst development efficiency of the cleaved embryos was compared, it was evident that the OPU-helper group (3+7), at 51.3%, was significantly higher than the 17.1% of the OPU embryo group (3+0) alone. When calculating the advantage of culturing along with agarose embedded helpers, the yield for OPU embryos reached a mean of 2.63 blastocysts/donor/session (7.1×37.1%) that represents an increase of 0.83 blastocysts/donor/session achieved over OPU embryos cultured alone (7.1×11.8%) (p<0.05).

As can be easily understood from the foregoing, the basic concepts of the present invention may be embodied in a variety of ways. The invention involves numerous and varied embodiments of shipping container and methods of making and using the shipping container including, but not limited to, the best mode of the invention.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of “separating” should be understood to encompass disclosure of the act of “separating”—whether explicitly discussed or not—and, conversely, were there effectively disclosure of the act of “separating”, such a disclosure should be understood to encompass disclosure of a “separator” and even a “means for separating.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

Moreover, for the purposes of the present invention, the term “a” or “an” entity refers to one or more of that entity; for example, “an embryo” refers to one or more of the embryos. As such, the terms “a” or “an”, “one or more” and “at least one” can be used interchangeably herein.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment.

Thus, the applicant(s) should be understood to claim at least: i) the methods disclosed and described for culturing embryos, ii) systems for separating embryos and for group culturing of embryos, iii) similar, equivalent, and even implicit variations of each of these systems and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

The claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A method of developing embryos comprising the steps of:
a. obtaining at least one embryo;
b. obtaining at least one helper embryo;
c. culturing the at least one embryo with the at least one helper embryo.

2. The method as claimed in claim 1, further comprising the step of:
a. maintaining separation between the at least one helper embryo and the at least one embryo during the step of culturing.

3. The method as claimed in claim 2, wherein the step of maintain separation further comprises embedding the at least one helper embryo in a gel or solid suspension and wherein the suspension is cultured with the at least one embryo.

4. The method as claimed in claim 3, wherein the at least one helper embryos provides a supporting and promoting effect on the at least one embryo while segregated.

5. The method as claimed in claim 4 wherein the suspension comprises an agarose chip.

6. The method as claimed in claim 5, wherein the step of embedding the at least one helper embryo further comprises the steps of
a. providing a solution containing agarose;
b. melting the solution;
c. adding at least one helper embryo to the melted solution; and
d. aspirating the at least one helper embryo and melted agarose solution to form a chip.

7. The method as claimed in claim 6 wherein the helper embryos are kept at about 30° C. to about 45° Celsius after being embedded.

8. The method as claimed in claim 2, wherein the at least one helper embryo influences the development of embryotrophic factors in the in the at least one embryo.

9. The method as claimed in claim 1, wherein the combined total embryos from at least one helper embryo and the at least one embryo is less than five.

10. The method as claimed in claim 1, wherein the combined total embryos from at least one helper embryo and the at least one embryo is less than five.

11. The method as claimed in claim 10, wherein a single helper embryo is incubated with a single embryo.

12. The method as claimed in claim 1 wherein the combined total embryos from at least one helper embryo and the at least one embryo is at least ten embryos.

13. The method as claimed in claim 12 wherein the combined total embryos from at least one helper embryo and the at least one embryo is at least twenty embryos.

14. The method as claimed in claim 13 wherein the combined total embryos from at least one helper embryo and the at least one embryo is at least forty embryos.

15. The method as claimed in claim 1 where the at least one embryo comprises one to nine embryos.

16. The method as claimed in claim 1 wherein the at least one embryo is formed from oocytes collected in an ultrasound assisted ovum pick up procedure.

17. The method as claimed in claim 1 wherein the at least one embryo is formed from oocytes collected from slaughterhouse ovaries.

18. The method as claimed in claim 1 wherein the helper embryos are formed from oocytes collected without reference to specific donors.

19. The method as claimed in claim 18 wherein the helper embryos are formed from oocytes collected from slaughterhouse ovaries.

20. The method as claimed in claim 1 wherein the helper embryos are formed from oocytes collected in an ultrasound assisted ovum pick up procedure

21. The method as claimed in claim 1 one wherein the at least one embryo comprises an oocyte fertilized in vitro with sex sorted sperm.

22. The method as claimed in claim 2 wherein the at least one embryo is separated from the helper embryos during culturing by a membrane.

23. The method as claimed in claim 22 wherein the membrane comprises permeable membrane across which fluids can cross.

24. The method as claimed in claim 23 wherein the fluid passage across the membrane provides a supporting and promoting effect on the at least one embryo while segregated from the helper embryos.

25. The method as claimed in claim 2 wherein the at least one embryo is segregated from the helper embryos by a barrier or membrane.

26. The method as claimed in claim 25 wherein the barrier comprises a mesh.

27. The method as claimed in claim 25 wherein the barrier comprises a porous structure.

28. The method as claimed in claim 25 wherein the barrier comprises a permeable barrier.

29. A method of developing embryos comprising the steps of:
a. collecting a first group of oocytes;
b. fertilizing the first group of oocytes to form a group of helper embryos;
c. collecting a second group of oocytes;
d. fertilizing the second group of oocytes with sex sorted sperm to form a group of sorted embryos; and
e. culturing the helper embryos with the sorted embryos wherein the groups of embryos are physically separated.

30. The method as claimed in claim 29 wherein the helper embryos are embedded in a gel or solid suspension and wherein the suspension is cultured with the at least one embryo.

31. The method as claimed in claim 29 wherein the suspension comprises an agarose chip.

32. The method as claimed in claim 29 wherein the helper embryos provide a supporting and promoting effect on the at least one embryo while segregated.

33. The method as claimed in claim 31 further comprising the steps of
a. providing a solution containing agarose;
b. melting the solution;
c. adding helper embryos to the melted solution; and
d. aspirating the helper embryos and melted agarose solution to form a chip.

34. The method as claimed in claim 29 wherein the helper embryos influence the development of embryotrophic factors in the in the at least one embryo.

35. The method as claimed in claim 29 wherein the combined total embryos from the helper embryos and the at least one embryo is at least ten embryos.

36. A culturing system comprising;
a. a space for incubating embryos; and
b. a plurality of helper embryos within the space.

37. The system of claim 36 wherein the space comprises at least one well and the plurality of helper embryos are located within the well.

38. The system of claim 37, wherein a plurality of helper embryos are embedded within each respective well.

39. The system of claim 37 wherein the helper embryos are embedded within in a permeable gel or a permeable solid in each well.

40. The system of claim 37 wherein the helper embryos are embedded in an agarose chip.

41. The system of claim 37 further comprising a membrane over the well.

42. The system of claim 37 wherein wells are divided into two portions.

43. The system of claim 42 wherein wells are divided by a membrane.

44. The system of claim 42 wherein wells are divided into concentric inner and outer areas.

45. The system of claim 42 further comprising an elevated portion.

46. The system of claim 45 further comprising a second well in the elevated portion for retaining cells.

47. The system of claim 46 wherein helper cells are embedded in the second well.

48. The system of claim 46 wherein helper cells are vertically separated into the second with membrane across the second well.

49. The system of claim 46 wherein at least one embryo is placed in the second well and is separated vertically from helper embryos by a membrane.

50. A method of embedding embryos in an agarose chip comprising the steps of:
a. producing a solution with agarose and NaCl;
b. autoclaving the solution;
c. heating the solution to melt the agarose;
d. cooling the solution to about a normal temperature for embryos;
e. adding embryos into the solution;
f. aspirating the embryos and agarose solution into an instrument with a channel; and
g. releasing the embryos and agarose solution on a cooler surface for solidifying the agarose.

51. The method according to claim 50 wherein the step of melting the agarose comprises heating the agarose to about 65° Celsius.

52. The method according to claim 50 wherein the embryos are added to the solution in the 2-8 cell stages of development.

53. The method according to claim 50 wherein the embryos are added at about 39° Celsius.

54. The method according to claim 50 wherein the embryos and agarose are released onto a medium between about 25° Celsius and about 30° Celsius.
